(19) United States
(12) Patent Application Publication
(10) Pub. No.: US 2007/0244544 A1
(43) Pub. Date: Oct. 18, 2007

(54) Seal for Enhanced Stented Valve Fixation

(75) Inventors: Matthew J. Birdsall, Santa Rosa, CA (US); Mark Jeffrey Dolan, Santa Rosa, CA (US); Justin Goshgarian, Santa Rosa, CA (US); Michael Krivoruchko, Forestville, CA (US); Joseph Francis Lessar, Coon Rapids, MN (US); Darrel Frank Untereker, Oak Grove, MN (US); Peter W. Urbanski, Maplewood, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/279,769

(22) Filed: Apr. 14, 2006

(51) Int. Cl. A61F 2/24; A61F 2/06

(52) U.S. Cl. 623/1.26; 623/1.36; 623/2.11; 623/2.38

(57) ABSTRACT

A valve replacement system that can be used for treating abnormalities of the right ventricular outflow tract includes a prosthetic valve device having a sealant contacting at least a portion of the outer surface of the valve device. The sealant may be breakable, and may be a hydrogel, an expandable hydrogel, or a solid. One embodiment of the invention includes a flowable sealant that is injected within the vascular system. Another embodiment of the invention includes a method for replacing a pulmonary valve that includes forming a seal around the exterior surface of a replacement valve and preventing blood flow around the replacement valve.

### TECHNICAL FIELD

This invention relates generally to medical devices for treating cardiac valve abnormalities, and particularly to a pulmonary valve replacement system and method of employing the same.

### BACKGROUND OF THE INVENTION

Heart valves, such as the mitral, tricuspid, aortic and pulmonary valves, are sometimes damaged by disease or by aging, resulting in problems with the proper functioning of the valve. Heart valve problems generally take one of two forms: stenosis, in which a valve does not open completely or the opening is too small, resulting in restricted blood flow; or insufficiency, in which blood leaks backward across a valve when it should be closed.

The pulmonary valve regulates blood flow between the right ventricle and the pulmonary artery, controlling blood flow between the heart and the lungs. Pulmonary valve stenosis is frequently due to a narrowing of the pulmonary valve or the pulmonary artery distal to the valve. This narrowing causes the right side of the heart to exert more pressure to provide sufficient flow to the lungs. Over time, the right ventricle enlarges, which leads to congestive heart failure (CHF). In severe cases, the CHF results in clinical symptoms including shortness of breath, fatigue, chest pain, fainting, heart murmur, and in babies, poor weight gain. Pulmonary valve stenosis most commonly results from a congenital defect, and is present at birth, but is also associated with rheumatic fever, endocarditis, and other conditions that cause damage to or scarring of the pulmonary valve. Valve replacement may be required in severe cases to restore cardiac function.

Previously, valve repair or replacement required open-heart surgery with its attendant risks, expense, and extended recovery time. Open-heart surgery also requires cardiopulmonary bypass with risk of thrombosis, stroke, and infarction. More recently, flexible valve prostheses and various delivery devices have been developed so that replacement valves can be implanted transvenously using minimally invasive techniques. As a consequence, replacement of the pulmonary valve has become a treatment option for pulmonary valve stenosis.

The most severe consequences of pulmonary valve stenosis occur in infants and young children when the condition results from a congenital defect. Frequently, the pulmonary valve must be replaced with a prosthetic valve when the child is young, usually less than five years of age. However, as the child grows, the valve can become too small to accommodate the blood flow to the lungs that is needed to meet the increasing energy demands of the growing child, and it may then need to be replaced with a larger valve. Alternatively, in a patient of any age, the implanted valve may fail to function properly due to calcium buildup and have to be replaced. In either case, repeated surgical or transvenous procedures are required.

To address the need for pulmonary valve replacement, various implantable pulmonary valve prostheses, delivery devices and surgical techniques have been developed and are presently in use. One such prosthesis is a bioprosthetic, valved conduit comprising a glutaraldehyde treated bovine jugular vein containing a natural, trileaflet venous valve, and sinus. A similar device is composed of a porcine aortic valve sutured into the center of a woven fabric conduit. A common conduit used in valve replacement procedures is a homograft, which is a vessel harvested from a cadaver. Valve replacement using either of these devices requires thoracotomy and cardiopulmonary bypass.

When the valve in the prostheses must be replaced, for the reasons described above or other reasons, an additional surgery is required. Because many patients undergo their first procedure at a very young age, they often undergo numerous procedures by the time they reach adulthood. These surgical replacement procedures are physically and emotionally taxing, and a number of patients choose to forgo further procedures after they are old enough to make their own medical decisions.

Recently, implantable stented valves have been developed that can be delivered transvenously using a catheter-based delivery system. These stented valves comprise a collapsible valve attached to the interior of a tubular frame or stent. The valve can be any of the valve prostheses described above, or it can be any other suitable valve. In the case of valves in harvested vessels, the vessel can be of sufficient length to extend beyond both sides of the valve such that it extends to both ends of the valve support stent.

The stented valves can also comprise a tubular portion or “stent graft” that can be attached to the interior or exterior of the stent to provide a generally tubular internal passage for the flow of blood when the leaflets are open. The graft can be separate from the valve and it can be made from any suitable biocompatible material including, but not limited to, fabric, a homograft, porcine vessels, bovine vessels, and equine vessels.

The stent portion of the device can be reduced in diameter, mounted on a catheter, and advanced through the circulatory system of the patient. The stent portion can be either self-expanding or balloon expandable. In either case, the stented valve can be positioned at the delivery site, where the stent portion is expanded against the wall of a previously implanted prostheses or a native vessel to hold the valve firmly in place.

One embodiment of a stented valve is disclosed in U.S. Pat. No. 5,957,949 titled “Percutaneous Placement Valve Stent” to Leonhardt, et al, the contents of which are incorporated herein by reference.

Although the use of stented valves can obviate the need for open heart surgery during installation, the stents are difficult to remove if replacement of the valve becomes necessary due to either the growth of the patient or calcification of the leaflets. Because the stent portion of the implantable valve is in tight contact with the vessel wall, it induces fibrosis in the surrounding vascular tissue, and is frequently infiltrated with tissue. To remove the stented valve, the stent portion must be cut from the vessel wall. This difficult procedure incurs a risk that the vessel wall will be punctured, and usually must be performed in an open surgical procedure.

It would be desirable, therefore, to provide an implantable pulmonary valve that can readily be replaced using minimally invasive surgical techniques, and would overcome the limitations and disadvantages inherent in the devices described above.

### SUMMARY OF THE INVENTION

It is an object of the present invention to provide a vascular valve replacement system having at least a delivery catheter and a replacement valve device disposed on the delivery catheter. The replacement valve device includes a prosthetic valve connected to a valve support region of an expandable support structure. The valve support region includes a plurality of protective struts disposed between a first stent region and a second stent region.

The system and the prosthetic valve will be described herein as being used for replacing a pulmonary valve. The pulmonary valve is also known to those having skill in the art as the “pulmonic valve” and as used herein, those terms shall be considered to mean the same thing.

Thus, one aspect of the present invention provides a system for treating abnormalities of the right ventricular outflow tract comprising a conduit, a catheter and a prosthetic valve device. The prosthetic valve device comprises a valve connected to a support structure and a sealant contacting at least a portion of the outer surface of the support structure of the valve device. When the valve device is deployed from the catheter and situated within the conduit, the sealant prevents blood flow between the inner wall of the conduit and the outer surface of the support structure of the valve device.

Another aspect of the invention provides a pulmonary valve replacement system comprising a conduit, a prosthetic valve device and a sealant. The valve device is positioned within the conduit and a flowable form of the sealant is deployed from a catheter. When the sealant is disposed about at least a portion of the outer surface of the support structure of the valve device, blood is prevented from flowing between the outer surface of the support structure of the valve device and the interior surface of the conduit.

Another aspect of the invention provides a pulmonary valve replacement system comprising a catheter, a prosthetic valve device and a moldable sealant. The system further comprises a molding device mounted on the catheter. The molding device comprises distal and proximal expandable seal portions that are spaced apart from each other so that the seal portions form an interior mold portion in the space between them. When a moldable sealant is positioned within the space between the seal portions and the interior wall of the conduit, it forms a symmetrical molded lumen to receive the valve device.

Another aspect of the invention provides a method for replacing a pulmonary valve. The method comprises using a catheter to deliver a pulmonary valve device to a treatment site. The pulmonary valve device includes a valve connected to a support structure and a sealant disposed about at least a portion of the outer surface of the support structure. The method further comprises deploying the valve device from the catheter, positioning the valve device within the conduit and forming a seal and thereby preventing blood flow around the support structure.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic interior view of a human heart showing the functioning of the four heart valves;

FIG. 2A is a schematic view showing the placement of a pulmonary conduit, as is known in the prior art;

FIG. 2B is a schematic view showing attachment of a pulmonary conduit to the pulmonary artery, as is known in the prior art;

FIG. 2C is a schematic view showing attachment of a pulmonary conduit to the heart, as is known in the prior art;

FIG. 3 is a diagram of a prosthetic pulmonary valve connected to a support structure with a sealant on the exterior surface of the support structure, in accordance with the present invention;

FIG. 4 is a schematic view of a prosthetic valve device situated in a conduit and a sealant composition being deployed from a catheter, in accordance with the present invention;

FIG. 5A is a schematic diagram of catheter having an expandable mold device in a conduit, in accordance with the present invention.

FIG. 5B is a schematic view of a moldable sealant that provides a symmetrical lumen to receive a prosthetic valve device, in accordance with the present invention; and

FIG. 6 is a flow diagram of a method of treating right ventricular outflow tract abnormalities by replacing a pulmonary valve, in accordance with the present invention.

### DETAILED DESCRIPTION

The invention will now be described by reference to the drawings wherein like numbers refer to like structures.

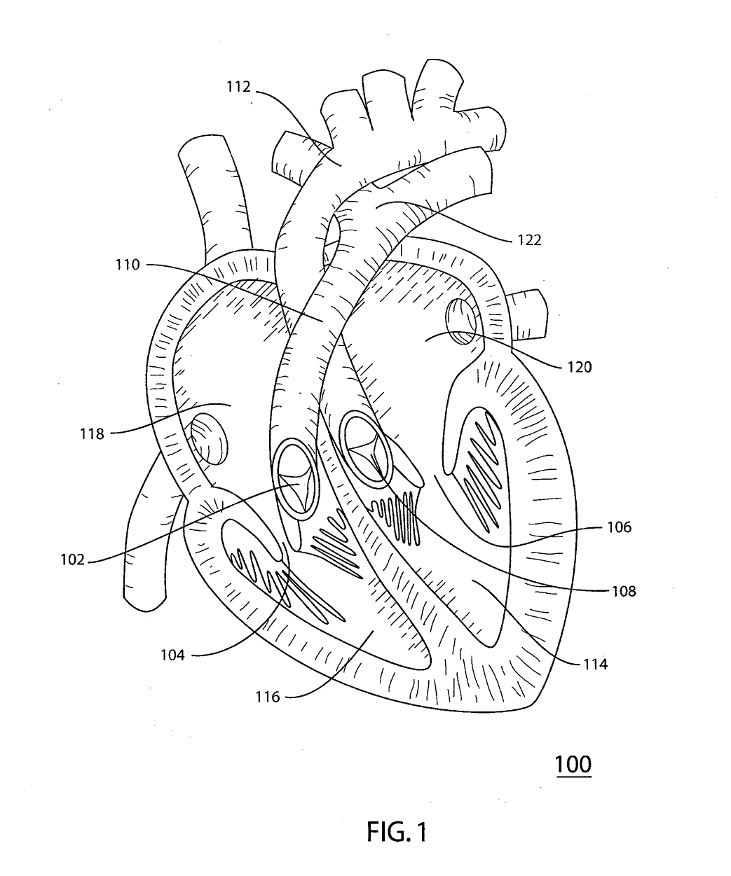

Referring to the drawings, FIG. 1 is a schematic representation of the interior of human heart 100. Human heart 100 includes four valves that work in synchrony to control the flow of blood through the heart. Tricuspid valve 104, situated between right atrium 118 and right ventricle 116, and mitral valve 106, between left atrium 120 and left ventricle 114 facilitate filling of ventricles 116 and 114 on the right and left sides, respectively, of heart 100. Aortic valve 108 is situated at the junction between aorta 112 and left ventricle 114 and facilitates blood flow from heart 100, through aorta 112 to the peripheral circulation.

Pulmonary valve 102 is situated at the junction of right ventricle 116 and pulmonary artery 110 and facilitates blood flow from heart 100 through the pulmonary artery 110 to the lungs for oxygenation. The four valves work by opening and closing in harmony with each other. During diastole, tricuspid valve 104 and mitral valve 106 open and allow blood flow into ventricles 114 and 116, and the pulmonic valve and aortic valve are closed. During systole, shown in FIG. 1, aortic valve 108 and pulmonary valve 102 open and allow blood flow from left ventricle 114, and right ventricle 116 into aorta 112 and pulmonary 110, respectively.

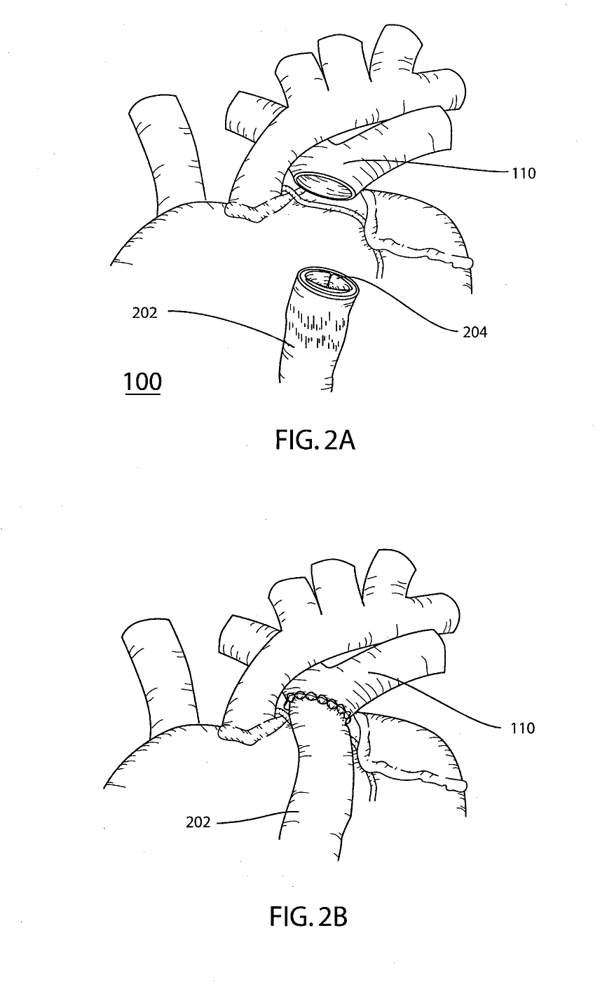

The right ventricular outflow tract is the segment of pulmonary artery 110 that includes pulmonary valve 102 and extends to branch point 122, where pulmonary artery 110 forms left and right branches that carry blood to the left and right lungs respectively. A defective pulmonary valve or other abnormalities of the pulmonary artery that impede blood flow from the heart to the lungs sometimes require surgical repair or replacement of the right ventricular outflow tract with prosthetic conduit 202, as shown in FIGS. 2A-C.

Such conduits comprise tubular structures of biocompatible materials, with a hemocompatible interior surface. Examples of appropriate biocompatible materials include polytetrafluoroethylene (PTFE), woven polyester fibers such as Dacron® fibers (E.I. Du Pont De Nemours & Co., Inc.), and bovine vein crosslinked with glutaraldehyde. One common conduit is a homograft, which is a vessel harvested from a cadaver and treated for implantation into a recipient's body. These conduits may contain a valve at a fixed position within the interior lumen of the conduit that functions as a replacement pulmonary valve.

One such conduit 202 comprises a bovine jugular vein with a trileaflet venous valve preserved in buffered glutaraldehyde. Other valves are made of xeno-pericardial tissue and are attached to the wall of the lumen of the conduit. Still other valves may be made at least partially from some synthetic material. The conduits may also include materials having a high X-ray attenuation coefficient (radiopaque materials) that are woven into or otherwise attached to the conduit, so that it can be easily located and identified.

As shown in FIGS. 2A and 2B, conduit 202, which houses valve 204 within its inner lumen, is installed within a patient by sewing the distal end of conduit 202 to pulmonary artery 110, and, as shown in FIG. 2C, attaching the proximal end of conduit 202 to heart 100 so that the lumen of conduit 202 connects to right ventricle 116.

Over time, implanted prosthetic conduits and valves are frequently subject to calcification, causing the affected conduit or valve to lose flexibility, become misshapen, and lose the ability to function effectively. Additional problems are encountered when prosthetic valves are implanted in young children. As the child grows, the valve will ultimately be too small to handle the increased volume of blood flowing from the heart to the lungs. In either case, the valve needs to be replaced.

The current invention discloses devices and methods for percutaneous catheter based placement of stented valves for regulating blood flow through a pulmonary artery. In a preferred embodiment, the valves are attached to an expandable support structure and they are placed in a valved conduit that is been attached to the pulmonary artery, and that is in fluid communication with the right ventricle of a heart. The support structure can be expanded such that any pre-existing valve in the conduit is not disturbed, or it can be expanded such that any pre-existing valve is pinned between the support structure and the interior wall of the conduit.

The delivery catheter carrying the stented valve is passed through the venous system and into a patient's right ventricle. This may be accomplished by inserting the delivery catheter into either the jugular vein or the subclavian vein and passing it through superior vena cava into right atrium. The catheter is then passed through the tricuspid valve, into right ventricle, and out of the ventricle into the conduit. Alternatively, the catheter may be inserted into the femoral vein and passed through the common iliac vein and the inferior vena cava into the right atrium, then through the tricuspid valve, into the right ventricle and out into the conduit. The catheters used for the procedures described herein may include radiopaque markers as are known in the art, and the procedure may be visualized using fluoroscopy, echocardiography, ultrasound, or other suitable means of visualization.

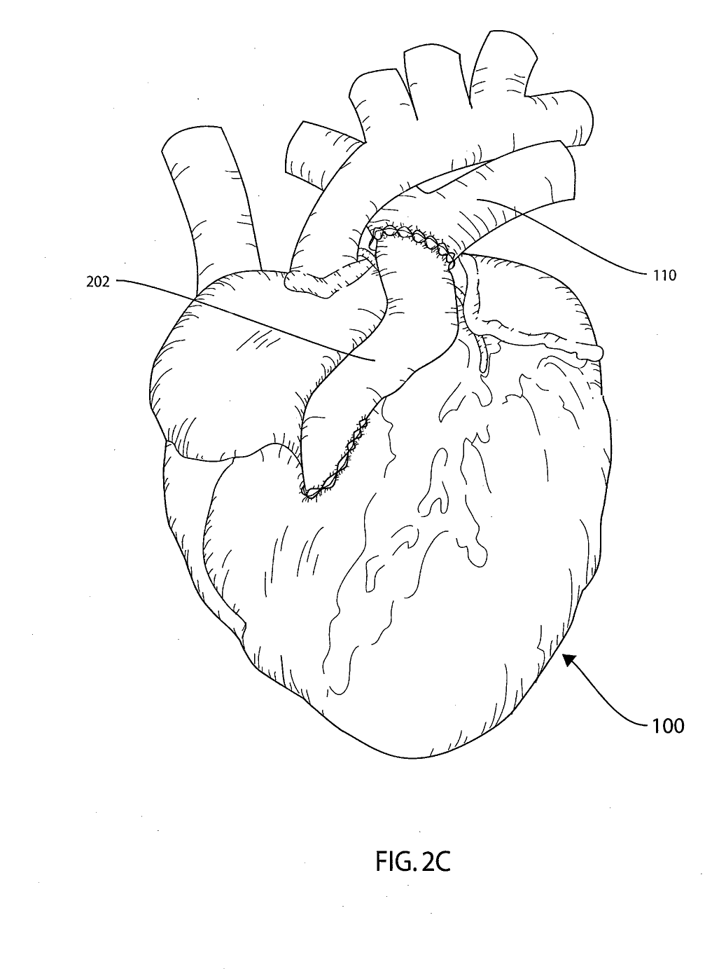

FIG. 3 is a cross-sectional side view of replacement valve device 300, in accordance with the present invention. Replacement valve 300 is suitable for use in either a prosthetic conduit such as conduit 202 or in pulmonary artery 110. Prosthetic valve 304 is situated within the lumen of expandable tubular support structure 302. In one embodiment of the invention, support structure 302 is a stent made of a flexible, biocompatible material that has “shape memory”, such as nitinol. Prosthetic valve 304 comprises three leaflets of a flexible material. The exterior surface of support structure 302 is coated with a sealant 306. In one embodiment of the invention sealant 306 is a hydrogel comprising one or more biostable polymers. The polymers include initiator and polymerizable chemical groups that react with each other and form a polymeric matrix that is insoluble in water. Alternatively, the hydrogel composition may include separate cross-linker molecules selected so that when the cross-linker is mixed with the polymer, the cross-linker reacts with chemical groups on the polymer molecules and a stable molecular network is formed. Suitable polymers include polyethylene glycol, polyvinyl alcohol, polyacrylamide, alginate, chitosan, and collagen. Polymer/cross-linker combinations include alginate combined with a divalent cation such as calcium or strontium, and derivatized polyethylene glycol in combination with cross-linker molecules with electrophilic or nucleophilic reactive groups, as is known in the art. The hydrogel retains water within the polymeric matrix and forms a soft, pliable mass that acts as a seal between the exterior surface of stent 302 and the interior wall of conduit 202. The polymeric hydrogel composition is applied to the exterior surface of stent 302 by spraying or dipping, as is well known in the art.

In one embodiment of the invention sealant 306 is an expandable hydrogel. Such hydrogel compositions are capable of undergoing hydration and dehydration. When exposed to water, in the at least partially dehydrated state, the expandable hydrogel composition absorbs water and the volume of the hydrogel increases. Polymers suitable for forming expandable hydrogels include: poly(ethylene oxide), poly(vinylpyrrolidone), polyvinyl alcohol, polyacrylamide, polyvinyl acetate, polyacrylic acid (Na⁺ form), poly(hydroxyethyl acrylate), poly(hydroxymethyl methacrylate), and hydrophilic poly(urethanes). Hydrogels comprising any such polymers alone or in combination are bound to the exterior surface of stent 302 in a partially dehydrated state. When device 300 is deployed in conduit 202 within the vascular system and exposed to blood, the hydrogel coating absorbs water, expands, and forms a tight seal between the exterior surface of stent 302 and the interior surface of conduit 202.

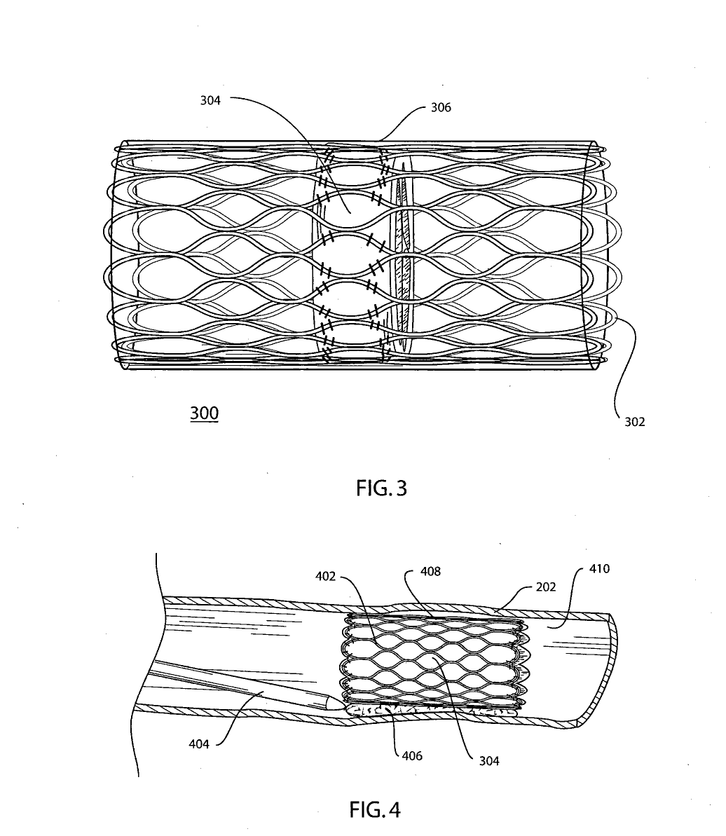

In one embodiment of the invention, the sealant composition is deployed from the catheter as a flowable liquid, and then forms a viscous hydrogel in situ within the lumen of conduit 202. As shown in FIG. 4, pulmonary valve replacement system 400 includes stented valve 402, deployed from catheter 404 and situated within lumen 410 conduit 202. If interior lumen 410 of conduit 202 is not symmetrical, exterior surface 408 of stented valve device 402 may not contact the interior wall of conduit 202, allowing blood to flow around valve device 402. In this embodiment of the invention, flowable composition 406 is delivered from the distal tip of catheter 404 and injected between exterior surface 408 of stented valve 402 and the interior surface of conduit 202. Flowable composition 406 then undergoes a physical or chemical change, and becomes a viscous hydrogel and forms a seal. Some compositions appropriate for this embodiment comprise a polymer and a cross-linker that are mixed within the tip of catheter 404, undergo a chemical reaction, and quickly form a hydrogel in situ. Other compositions comprise polymers having photoreactive groups that are activated by exposure to light of a specific wavelength, and form bonds between the polymer molecules to produce a hydrogel. In this embodiment of the invention, the distal tip of catheter 404 includes a fiber optic light source of the required wavelength. Still other polymer compositions change viscosity in response to temperature changes, and may be applied as liquids and form hydrogels in situ as they approach body temperature.

In one embodiment of the invention, flowable liquid 406 becomes a solid upon exposure to blood and forms a seal between the interior wall of conduit 202 and stented valve 402. One such polymer composition is cyanoacrylate dissolved in dimethylsulfoxide. When injected into the circulatory system, the dimethylsulfoxide is diluted and removed in the flowing blood, and the cyanoacrylate precipitates and forms a solid barrier that prevents blood flow around stented valve 402.

Besides hydrogels and solids, other embodiments of the invention include sealants that comprise gums, pastes, or other materials that are malleable and form a seal in an aqueous environment and prevent blood flow between a device such as stented valve 402 and the interior wall of conduit 202.

Sometimes prosthetic valves become calcified and need to be replaced. In one embodiment of the invention, either sealant 306 or sealant 406 forms a breakable seal between the exterior surface of stented valve 302 or 402 respectively. An example of this embodiment is a hydrogel sealant comprising alginate polymers cross-linked with divalent calcium ions. This hydrogel provides a seal that is stable and sufficiently robust to prevent blood flow around valve 302 or 402, but is pliable, and will break in response to a minimum amount of force applied to it, and allow stented valve 302 or 402 to be dislodged and removed. The alginate hydrogel may be coated on the exterior surface of stented valve 302 and form hydrogel sealant 306. Alternatively, a flowable alginate composition can be mixed with a solution of calcium ions and immediately injected between the interior wall of conduit 202 and the exterior surface of stented valve 402. In either case the calcium/alginate hydrogel will provide a breakable seal.

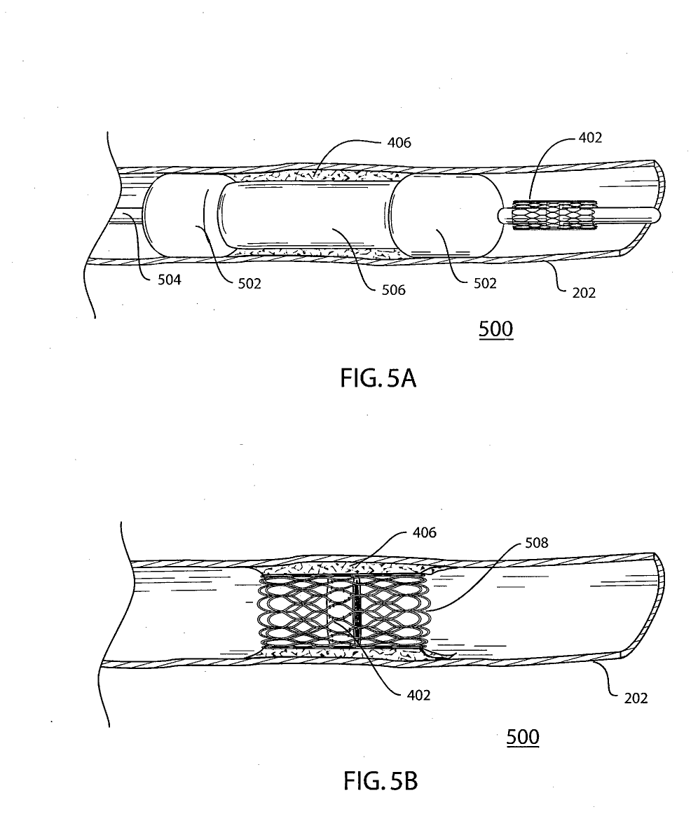

Conduit 202 is a long term implant and it can become calcified or be subject to fibrotic ingrowth of tissue, either of which can cause conduit 202 to become misshapen, so that its cross section is no longer round and symmetrical. Consequently, stented valve 402 does not fit well within conduit 202, and may be ineffective either because of blood flowing around the outside of stented valve 402, or because valve 402 cannot be aligned perpendicularly to the flow of blood through conduit 202. FIG. 5A is a schematic representation of system 500 for replacing a pulmonary valve in either a misshapen blood vessel or a portion of conduit 202 that is not symmetrical, in accordance with the present invention. Two inflatable balloons 502 are mounted in the distal portion of catheter 504. Balloons 502 are spaced apart from each other so that, when inflated, they block blood flow through conduit 202, and form a space between them. In one embodiment of the invention, catheter 504 is a perfusion catheter, having a lumen that carries blood through the blocked portion of conduit 202 while balloons 502 are inflated. Between inflatable balloons 502, and parallel to the body of catheter 504, is a third inflatable balloon 506. When inflated, the diameter of the outer surface of balloon 506 is substantially the same as the outer diameter of stented valve 402, and forms a central lumen through the molded hydrogel mass. Thus, the space between inflated balloons 502 and around balloon 506 forms a tubular mold in the interior of conduit 202. The exterior surfaces of balloons 502 and 506 may be coated with a release agent such as silicone or polytetrafluoroethylene (PTFE) to prevent the hydrogel mass from adhering to balloons 502 and 506. Flowable composition 406 is then delivered from a distal portion of catheter 504, and fills the tubular mold. In this embodiment of the invention, flowable composition 406 forms a moldable hydrogel mass 406 that adheres to the interior wall of conduit 202, and has sufficient mechanical strength to maintain its shape after it is delivered, and to hold the stented valve in a fixed position oriented parallel to the direction of blood flow through conduit 202. Biostable polymers suitable for forming moldable hydrogels include polyalkenes, polyesters, polyacrylates, polymethacrylates, polyamides and polysaccharides.

Once the moldable hydrogel has formed a firm mass, balloons 502 and 506 are deflated and catheter 504 is partially withdrawn so that stented valve 402 is situated within lumen 508 (FIG. 5B) of gel mass 406. Stented valve 402 is then deployed from catheter 504, and catheter 504 is withdrawn from the body. Hydrogel mass 406 provides a firm support for stented valve 402 that maintains stented valve 402 in a fixed position and orientation by maintaining a uniform distribution of stress loads along the length of the stent.

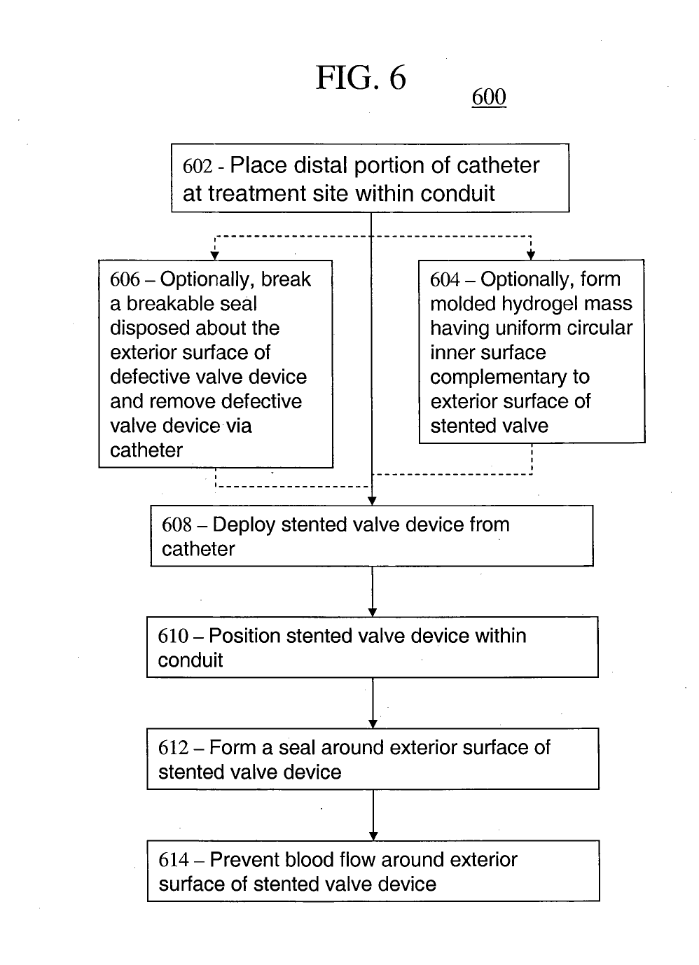

FIG. 6 is a flowchart illustrating method 600 for treating right ventricular outflow tract abnormalities by replacing a pulmonary valve, in accordance with the present invention. The distal portion of delivery catheter 504 is inserted into the vascular system of the patient, and is then passed through the venous system and into a patient's right ventricle 116. This may be accomplished by inserting delivery catheter 504 into either the jugular vein or the subclavian vein, and passing it through the superior vena cava into right atrium 118. The catheter is then passed through tricuspid valve 104, into right ventricle 116, and out of the ventricle into either conduit 202 or the pulmonary artery. Alternatively, delivery catheter 504 may be inserted into the femoral vein and passed through the common iliac vein and the inferior vena cava into right atrium 118, then through tricuspid valve 104, into right ventricle 116, and out into conduit 308. The catheters used for the procedures described herein may include radiopaque markers as is known in the art, and the procedure may be visualized using fluoroscopy, echocardiography, ultrasound, or other suitable means of visualization. The distal portion of delivery catheter 504 is then positioned at the treatment site within conduit 202, as indicated in Block 602.

In one embodiment of the invention, a flowable form of hydrogel 406 is delivered from catheter 504 and molded so that hydrogel 406 forms a mass having a uniform, circular inner surface complementary to the exterior surface of stented valve device 402, as indicated in Block 604. In another embodiment of the invention, a breakable hydrogel seal disposed about the exterior surface of stented valve device 302 or 402 is broken and stented valve 302 or 402 is dislodged and removed (Block 606).

Next, stented valve device 302 or 402 is deployed from catheter 504 (Block 608), and positioned within conduit 202 (Block 610). Stented valve device 302 or 402 is delivered to the conduit 202 or vessel in a collapsed state. Stented valve 302 or 402 expands upon deployment form the catheter. As indicated in Block 612, a seal is then formed around the exterior surface of either stented valve device 302 or 402. In the case of stented valve device 302, a sealant is disposed about at least a portion of the exterior surface. The sealant, for example a hydrogel, contacts the interior wall of conduit 202 and forms a seal. In one embodiment of the invention, the hydrogel sealant absorbs water and expands so that it contacts the wall of conduit 202 and forms a seal. Stented valve device 402 is positioned within conduit 202, and a flowable sealant is injected between the exterior of stented valve device 402 and the wall of conduit 202. The sealant forms either a hydrogel or a solid and forms a seal. In any of the above embodiments, the seal around the exterior of device 302 or 402 prevents blood flow around the exterior surface of the stented valve device, as indicated in Block 614.

While the invention has been described with reference to particular embodiments, it will be understood by one skilled in the art that variations and modifications may be made in form and detail without departing from the spirit and scope of the invention.

1. A vascular valve replacement system, the system comprising:
a conduit having a lumen;
a catheter;
a prosthetic valve device including a valve connected to an expandable support structure, the valve device disposed on the catheter; and
a sealant disposed about an outer surface of the support structure of the valve device wherein when the prosthetic valve device is deployed from the catheter within the lumen of the conduit and the support structure is expanded, the sealant prevents blood flow between the conduit and the outer surface of the support structure of the valve device.

2. The system of claim 1 wherein the sealant is a hydrogel.

3. The system of claim 2 wherein the hydrogel comprises a biocompatible, nonthrombogenic polymer or copolymer composition in an aqueous medium.

4. The system of claim 3 wherein the polymer composition comprises polymers or copolymers that are cross-linked using a cross-linking agent.

5. The system of claim 4 wherein the polymer is alginate and the cross-linking agent is a divalent cation.

6. The system of claim 5 wherein the cross-linked alginate forms a breakable seal that prevents fibrosis about the exterior surface of the support structure and thereby facilitates removal of the valve device.

7. The system of claim 3 wherein the polymer composition is capable of hydration and dehydration.

8. The system of claim 7 wherein the volume of the hydrogel increases upon hydration of the polymer composition.

9. A pulmonary valve replacement system, the system comprising:
a conduit having an interior wall forming a lumen;
a prosthetic valve device including a valve connected to a support structure, the valve device positionable in the lumen of the conduit, and
a sealant composition disposed about at least a portion of the outer surface of the support structure, wherein the sealant composition is deployed in a flowable form via a catheter to prevent blood flow between the interior wall of the conduit and the outer surface of the support structure of the valve device.

10. The system of claim 9 wherein the sealant composition forms a hydrogel within the lumen of the conduit.

11. The system of claim 10 wherein the sealant composition comprises at least one polymer or copolymer and a cross-linking agent wherein when the polymer or copolymer is mixed with the cross-linking agent the composition forms a hydrogel.

12. The system of claim 9 wherein the sealant composition becomes a solid within the lumen of the conduit.

13. The system of claim 12 wherein the sealant composition comprises at least one polymer or copolymer that precipitates upon exposure to biological fluids.

14. A pulmonary valve replacement system, the system comprising:
a catheter;
a prosthetic valve device including a valve connected to an expandable support structure, the valve device disposed on the catheter;
an expandable molding device disposed on the catheter, the molding device including spaced-apart, distal and proximal seal portions and an interior mold portion positioned between the distal and proximal seal portions, and
a moldable sealant positioned within a space between an outer surface of the mold portions and an interior conduit wall to form a symmetrical molded lumen to receive the valve device.

15. The system of claim 14 wherein in a solid state, the moldable sealant exerts uniform pressure on all areas of the exterior surface of the support structure and maintains the prosthetic valve device in a fixed position that is perpendicular to the direction of blood flow within a vascular conduit.

16. The system of claim 15 wherein, in the solid state, the moldable sealant forms a seal and prevents blood flow around the support structure.

17. A method for replacing a valve, the method comprising:
delivering a prosthetic valve device including a valve connected to a support structure having a sealant disposed about at least a portion of the outer surface of the support structure to a treatment site within a conduit via catheter;
deploying the prosthetic valve device from the catheter;
positioning the prosthetic valve device within a conduit;
forming a seal; and thereby preventing blood flow around the support structure via the sealant.

18. The method of claim 17 wherein forming a seal further comprises cross-linking at least one polymer or copolymer in an aqueous solution and forming a hydrogel in situ within the conduit.

19. The method of claim 17 wherein forming a seal further comprises precipitating at least one polymer or copolymer upon exposure to biological fluids within the conduit.

20. The method of claim 17 wherein forming a seal further comprises increasing the volume of a hydrogel by hydrating the polymer composition comprising the hydrogel and thereby increasing the volume of the hydrogel.

21. The method of claim 17 wherein forming a seal further comprises expressing a flowable composition from the catheter adjacent to the exterior surface of the support structure at the treatment site within the conduit and thereby preventing blood flow around the support structure via the flowable composition.

22. The method of claim 21 wherein forming a seal further comprises
molding the flowable composition so that it forms a mass that adheres to the vessel wall and has a uniform circular inner surface complementary to the exterior surface of the support structure;
solidifying the flowable composition;
deploying the prosthetic valve and support structure from the catheter;
inserting the prosthetic valve and support structure into mass of the solidified composition;
forming a seal around the support structure and valve; and
maintaining the prosthetic valve in a fixed position that is perpendicular to the direction of blood flow within the vascular conduit.

23. The method of claim 17 wherein replacing a pulmonary valve further comprises removing a defective prosthetic valve connected to the support structure by breaking a breakable seal disposed about the exterior surface of the support structure and removing the defective valve and support structure via catheter.
